(19) United States
(12) Patent Application Publication
(10) Pub. No.: US 2022/0042191 A1
(43) Pub. Date: Feb. 10, 2022

(54) HYDROGEN FLOW AND RATIO CONTROL USING ELECTROLYZER STACK CURRENT

(71) Applicant: Alliance for Sustainable Energy, LLC, Golden, CO (US)

(72) Inventors: Kevin William HARRISON, Erie, CO (US); Nancy Sue FARMER, Arvada, CO (US)

(21) Appl. No.: 17/397,665

(22) Filed: Aug. 9, 2021

Related U.S. Application Data
(60) Provisional application No. 63/063,000, filed on Aug. 7, 2020.

(51) Int. Cl. C25B 15/029; C25B 1/04; C25B 9/19; C25B 15/08; C12M 1/107; C12M 1/34; G01N 33/18

(52) U.S. Cl. CPC C25B 15/029; C25B 1/04; C25B 9/19; G01N 33/18; C12M 21/04; C12M 41/34; C25B 15/081

(57) ABSTRACT

Disclosed herein are methods, devices and systems to control hydrogen flow and ratios by using electrolyzer stack current to control to gas flowrate and ratio control through using only an electrolyzer stack current to control the H₂ mass flow rate to downstream processes requiring precise ratio control of the H₂ input gas with other process inputs, like CO₂, for example. In an embodiment, the systems disclosed herein deliver a precise ratio control to maintain a stable reaction and minimize excess H₂ and CO₂ in the product

### CROSS-REFERENCE TO RELATED APPLICATIONS

This application claims priority under 35 U.S.C. § 119 to U.S. Provisional Patent Application No. 63/063,000, filed Aug. 7, 2020, the contents of which are hereby incorporated by reference in its entirety.

### CONTRACTUAL ORIGIN

The United States Government has rights in this invention under Contract No. DE-AC36-08GO28308 between the United States Department of Energy and Alliance for Sustainable Energy, LLC, the Manager and Operator of the National Renewable Energy Laboratory.

### BACKGROUND

Hydrogen mass transfer can be the limiting input to many processes due to its inherent low solubility in water and low density. Downstream processes that rely on a hydrogen/carbon ratio near stoichiometry struggle with state-of-the-art Coriolis mass flow sensors due to hydrogen's low density. In addition, any liquid that condenses out of the hydrogen gas stream inside the mass flow sensor results in unreliable gas monitoring.

### SUMMARY

In an aspect, disclosed herein is a device for measuring the amount of hydrogen in a solution wherein the device measures the direct current output from an electrolyzer that separates water into hydrogen and oxygen and wherein the device measures the direct current output generated by a power supply and wherein the device uses Equation 1 to provide the amount of hydrogen in a solution. In an embodiment, the device does not use a Coriolis mass flowmeter. In an embodiment, the electrolyzer comprises a polymer electrolyte membrane.

In an aspect disclosed herein is a system comprising a bioreactor, a power supply, an electrolyzer and a device for measuring the amount of hydrogen in a solution wherein the device measures the direct current output from an electrolyzer that separates water into hydrogen and oxygen and wherein the device measures the direct current output generated by a power supply and wherein the device uses Equation 1 to provide the amount of hydrogen in a solution. In an embodiment, the bioreactor comprises a biocatalyst that uses carbon dioxide and hydrogen to generate methane, water and heat and wherein the amount of hydrogen in the solution is determined and controlled by using the device and wherein the amount of hydrogen in the solution is changed depending upon the amount of carbon dioxide in the bioreactor in order to maximize the amount of methane produced by the bioreactor. In an embodiment, the device does not use a Coriolis mass flowmeter. In an embodiment, the electrolyzer comprises a polymer electrolyte membrane. In an embodiment, the system further comprises a water and oxygen phase separator. In an embodiment, the system further comprises a flow control valve. In an embodiment, the flow of hydrogen and water is from the cathode side of the electrolyzer to the input for the bioreactor.

In an aspect, disclosed herein is a method for controlling the amount of dissolved hydrogen fed to a bioreactor wherein the method comprises a device that measures the direct current output from an electrolyzer that separates water into hydrogen and oxygen and wherein the device measures the direct current output generated by a power supply and wherein the device uses Equation 1 to provide the amount of hydrogen in a solution and wherein the amount of hydrogen in a solution measured by the device is compared to the amount of a product being produced by the bioreactor and wherein the amount of direct current provided to the electrolyzer is adjusted to alter the amount of hydrogen fed to the bioreactor. In an embodiment, the device does not use a Coriolis mass flowmeter. In an embodiment, the electrolyzer comprises a polymer electrolyte membrane. In an embodiment, the flow of hydrogen and water is from the cathode side of the electrolyzer to the input for the bioreactor. In an embodiment, the bioreactor produces methane.

Other objects, advantages, and novel features of the present invention will become apparent from the following detailed description of the invention when considered in conjunction with the accompanying drawings.

### BRIEF DESCRIPTION OF THE DRAWING

FIG. 1 depicts a process flow diagram embodiment of the mass flow control device that simplifies and improves process control and reliability between a hydrogen-producing electrolyzer stack and a downstream process using carbon dioxide as a feedstock with hydrogen to produce methane (CH₄).

### DETAILED DESCRIPTION

Electrolyzer systems use direct current (DC) to separate water molecules into its two constituent parts; hydrogen (H₂) and oxygen (O₂). This direct current is monitored between the power supply and the electrolyzer stack and provides a direct relationship to the amount of hydrogen (H₂) gas being produced (Equation 1).

Mass Flow_{H2},wet[g/sec]=(Istack/2F)*nMH₂ηFaraday Equation 1

Istack—Electrolyzer stack current (ADC in Coulomb per second)

F—Faraday's Constant (Coulombs per mol of electrons)

2—Number of mol of electrons per mol of H₂

n—Number of cells in the stack

M_{H2}—Molar mass of Hydrogen (g/mol)

ηFaraday—Faraday efficiency assumed to be 0.99 for PEM electrolysis

When an industrial process requires precise gas flow monitoring and control, flowmeters based on the Coriolis Effect are most often selected due to their accuracy. However, Coriolis mass flowmeters lose accuracy with lower density gases like H₂ even when gas pressure is elevated to increase their density. These flowmeters typically cost about $10,000-$12,000. Furthermore, any moisture that condenses out of the gas stream or finds its way to the flowmeter interferes with its ability to monitor mass flow accurately.

Previous Coriolis meters are not intended to be used in multiphase fluids, specifically mixtures of liquids and gases. If there is liquid vapor in the gas, this will affect the mass flow and the meter will read high.

Using systems and devices as disclosed herein eliminates the need for complex proportion flow control valves to maintain precise H₂ flowrate for input gas ratio control.

Electrolyzer stacks, including polymer electrolyte membrane (PEM), alkaline and solid oxide, are electrochemical devices used to produce hydrogen and oxygen gas for numerous applications. Due to the ion-transporting physics behind these reactions, these electrolyzer stacks respond to changes in current nearly instantaneously. This fast response allows the electrolyzer system to adapt the H₂ production rate very quickly to accommodate changes in other inputs to downstream processes such as those using CO₂.

System-level controls can adjust electrolyzer stack current directly thus changing the H₂ production rate instantaneously to adapt to downstream process upsets and changes that cause variations in gas or other inputs. Electrolyzer stack current is monitored between the DC power supply and the electrolyzer stack, so would not represent an additional cost to the system. Besides being needed for normal stack health monitoring, control and safety, the stack current sensor value can be used to calculate H₂ flow to maintain the gas ratio control between H₂ being produced at the electrolyzer and other input gases.

In subsequent downstream systems that require ratio control of hydrogen to other input gases, like carbon dioxide (CO₂), stack current can be used to deliver the exact stoichiometric flowrates to the process. Hydrogen mass transfer can be the limiting input to many processes due to its inherent low solubility in water and low density.

For example, the biological upgrading of CO₂ using H₂ and methanogens requires 4 molecules of H₂ for every molecule of CO₂. These gas inputs result in each CO₂ molecule being converted to methane (CH₄), two water (H₂O) molecules and heat described in equation 2.

CO₂+4H₂(+)Biocatalyst=CH₄+2H₂O+heat Equation 2

In practice, however, these systems typically operate at slightly elevated Hydrogen-to-Carbon (H/C) ratios in the 4.05-4.3 range to ensure that the process doesn't suffer from insufficient H₂ gas, which is in many cases the reaction-limiting input.

Thus, in an embodiment, the present disclosure has benefits over state-of-the-art solutions to gas flowrate and ratio control of using only an electrolyzer stack current to control the H₂ mass flow rate to downstream processes requiring precise ratio control of the H₂ input gas with other process inputs, like CO₂, for example. The systems disclosed herein deliver a precise ratio control to maintain a stable reaction and minimize excess H₂ and CO₂ in the product.

In an embodiment, electrolyzer stack current is controlled and responds nearly instantaneously (<3 seconds) to respond to changes in the process requiring gas flowrate adjustment for stable operations and recovery.

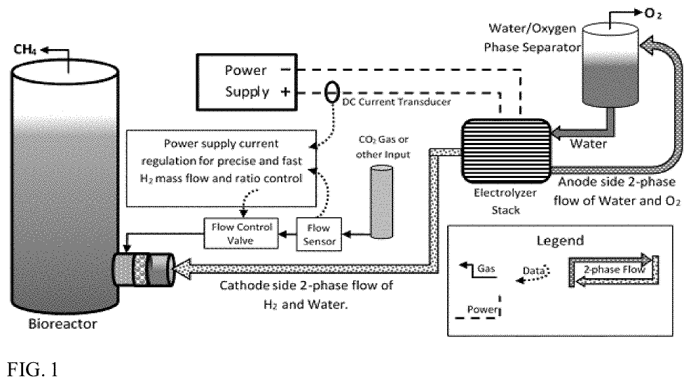

In an embodiment, the systems disclosed herein eliminate the need for costly H₂ mass flow monitoring and control at the input of these downstream processes, see FIG. 1.

The approach of utilizing the H₂ dissolved in water on the cathode side of the electrolyzer stack can improve H₂ mass transfer to downstream processes, for example those that are water-based such as biomethanation.

However, the extra hydrogen-rich water added to the downstream process may dilute nutrients and organism concentrations leading to reduced productivity and higher operational costs. These potential setbacks can be avoided if nutrient recovery and organism retention systems are in place. An additional benefit to systems disclosed herein is that the extra water may also be recovered, cleaned and used by the electrolyzer system again.

In an embodiment, disclosed herein is a gas-exchange membrane directly coupled to the downstream process such as a bioreactor. In an embodiment, the two-phase flow from the cathode of the electrolyzer stack is directly connected to the membrane separating the biomethanation process fluid from the water and hydrogen coming from the electrolyzer stack.

The passive membrane allows hydrogen gas to transfer to the bioreactor process fluid while keeping out most of the excess water coming from the electrolyzer stack. However, the water from the stack, which has dissolved H₂ gas in it is optimized to cover part or all of the membrane to allow transfer of this additional H₂ as well.

Thus in an embodiment, methods are disclosed herein that allow for precise ratio control to maintain stable organism productivity and minimize excess H₂ and CO₂ in the product.

The foregoing disclosure has been set forth merely to illustrate the invention and is not intended to be limiting.

We claim:

1. A device for measuring the amount of hydrogen in a solution wherein the device measures the direct current output from an electrolyzer that separates water into hydrogen and oxygen and wherein the device measures the direct current output generated by a power supply and wherein the device uses Equation 1 to provide the amount of hydrogen in a solution.

2. The device of claim 1 that does not use a Coriolis mass flowmeter.

3. The device of claim 1 wherein the electrolyzer comprises a polymer electrolyte membrane.

4. A system comprising a bioreactor, a power supply, an electrolyzer and a device for measuring the amount of hydrogen in a solution wherein the device measures the direct current output from an electrolyzer that separates water into hydrogen and oxygen and wherein the device measures the direct current output generated by a power supply and wherein the device uses Equation 1 to provide the amount of hydrogen in a solution.

5. The system of claim 4 wherein the bioreactor comprises a biocatalyst that uses carbon dioxide and hydrogen to generate methane, water and heat and wherein the amount of hydrogen in the solution is determined and controlled by using the device and wherein the amount of hydrogen in the solution is changed depending upon the amount of carbon dioxide in the bioreactor in order to maximize the amount of methane produced by the bioreactor.

6. The system of claim 4 wherein the device does not use a Coriolis mass flowmeter.

7. The system of claim 4 wherein the electrolyzer comprises a polymer electrolyte membrane.

8. The system of claim 4 further comprising a water and oxygen phase separator.

9. The system of claim 4 further comprising a flow control valve.

10. The system of claim 4 wherein the flow of hydrogen and water is from the cathode side of the electrolyzer to the input for the bioreactor.

11. A method for controlling the amount of dissolved hydrogen fed to a bioreactor wherein the method comprises a device that measures the direct current output from an electrolyzer that separates water into hydrogen and oxygen and wherein the device measures the direct current output generated by a power supply and wherein the device uses Equation 1 to provide the amount of hydrogen in a solution and wherein the amount of hydrogen in a solution measured by the device is compared to the amount of a product being produced by the bioreactor and wherein the amount of direct current provided to the electrolyzer is adjusted to alter the amount of hydrogen fed to the bioreactor.

12. The method of claim 11 wherein the device does not use a Coriolis mass flowmeter.

13. The method of claim 11 wherein the electrolyzer comprises a polymer electrolyte membrane.

14. The method of claim 11 wherein the flow of hydrogen and water is from the cathode side of the electrolyzer to the input for the bioreactor.

15. The method of claim 11 wherein the bioreactor produces methane.
